(19) United States
(12) Patent Application Publication
(10) Pub. No.: US 2005/0251198 A1
(43) Pub. Date: Nov. 10, 2005

(54) Intravascular filter membrane and method of forming

(75) Inventors: Mark S. Smith, Coon Rapids, MN (US); James G. Hansen, Minneapolis, MN (US); Justin M. Crank, Maple Grove, MN (US); Narin Anderson, Savage, MN (US); Horng-Ban Lin, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SciMed Life Systems, Inc.

(21) Appl. No.: 10/841,684

(22) Filed: May 6, 2004

(51) Int. Cl. A61M 29/00

(52) U.S. Cl. 606/200

(57) ABSTRACT

Intravascular filters formed by a molding process can have a plurality of integrally formed apertures. A molding process can utilize a mold assembly that includes a mold having a mold surface and a die having a die surface. The mold assembly includes plurality of protrusions that extend from at least one of the mold surface and the die surface. A molten material is placed within a portion of the mold, and the die is then inserted into the mold such that the plurality of protrusions span a distance between the die surface and the mold surface. The molten material is allowed to solidify, thereby forming a filter membrane that includes a plurality of integrally formed apertures.

### TECHNICAL FIELD

The invention relates generally to intravascular filter membranes and methods of their formation. In particular, the invention relates to methods of molding intravascular filter membranes having a plurality of integrally formed apertures.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic or narrowed blood vessels may be treated with a number of relatively non-invasive medical procedure including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated, and the restriction in the vessel is opened. During an atherectomy procedure, the stenotic lesion may be mechanically or otherwise cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel.

Because of this debris, a number of devices, such as intravascular filters, have been developed to filter out debris. A need remains for improved intravascular filters and filter membranes. A need remains for improved methods of manufacture of intravascular filters and filter membranes.

### SUMMARY

The present invention is directed to methods of molding intravascular filter membranes, the resulting intravascular filter membranes having a plurality of integrally formed apertures, and filters utilizing such filter membranes.

Accordingly, an example embodiment of the invention can be found in a method of forming a filter membrane using a mold assembly. The mold assembly includes a mold having a mold surface and a die having a die surface. The mold assembly includes a plurality of protrusions that extend from at least one of the mold surfaces or the die surface. A molten material is placed within a portion of the mold, and the die is then inserted into the mold such that the protrusions span a distance between the die surface and the mold surface. The molten material is allowed to solidify, thereby forming a filter membrane that includes a plurality of integrally formed apertures.

Another example embodiment of the invention can be found in an assembly adapted for forming a filter membrane. The assembly includes a mold having a mold surface that defines an at least partially conical cavity. A plurality of protrusions extend outwardly from the mold surface, each of the protrusions having a protrusion length. The assembly also includes a die that has a die surface that is complementary to the mold surface and is configured such that when the die is inserted into the mold, the protrusions extending from the mold surface contact the die surface.

Another example embodiment of the invention can be found in a filter membrane that is formed by a particular process. A mold having a mold surface and a plurality of protrusions extending outwardly from the mold surface is provided. A complementary die having a die surface is also provided. A molten material is provided within a portion of the mold and the die is extended into the mold such that the protrusions contact the die surface. The molten material is allowed to solidify, thereby forming a filter membrane having a plurality of integrally formed apertures.

Another example embodiment of the invention can be found in a filter assembly that includes a support loop and a filter membrane having a proximal region and a distal region. The support loop is integrally molded into the proximal region of the filter membrane, and the filter membrane includes a plurality of integrally formed apertures. A distal waist is integrally molded into the distal region of the filter membrane.

### BRIEF DESCRIPTION OF THE DRAWINGS

The invention may be more completely understood in consideration of the following detailed description of various embodiments of the invention in connection with the accompanying drawings, in which:

FIG. 1 is a schematic perspective view of an intravascular filter in accordance with an embodiment of the invention;

FIG. 2 is a magnified view of a portion of the filter membrane included in the intravascular filter of FIG. 1;

FIG. 3 is a cutaway view of a mold and die assembly in accordance with an embodiment of the invention;

FIG. 4 is a cutaway view of a mold and die assembly in accordance with an embodiment of the invention;

FIG. 5 is a cutaway view of a mold and die assembly in accordance with an embodiment of the invention;

FIG. 6 is a cutaway view of a mold in accordance with an embodiment of the invention;

FIG. 7 is a cutaway view of the mold of FIG. 6, with the inclusion of molten material;

FIG. 8 is a cutaway view of the mold of FIG. 7, with a complementary die extended into the mold;

FIG. 9 is a perspective view of an intravascular filter membrane produced in accordance with the exemplary process shown in FIGS. 6 through 8;

FIG. 10 is a cutaway view of a two-piece mold in accordance with an embodiment of the invention;

FIG. 11 is a cutaway view of a mold and die assembly as in FIG. 8, with the inclusion of a support loop positioned within the mold;

FIG. 12 is a perspective view of the intravascular filter membrane with an integral support loop produced in the mold and die assembly shown in FIG. 11; and

FIG. 13 is a cutaway view of a mold and die assembly in accordance with an embodiment of the invention.

### DETAILED DESCRIPTION OF THE PREFERRED EMBODIMENTS

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

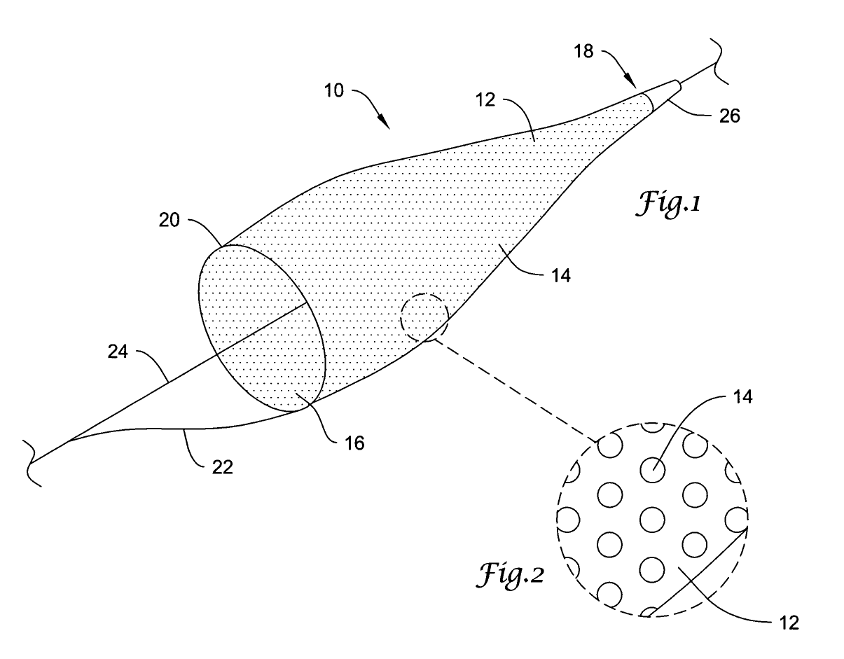

FIG. 1 is a perspective view of an example intravascular filter 10, which includes a filter membrane 12. The filter membrane 12 can be formed from any suitable moldable material or combination of materials. For example, the filter membrane 12 can include polymers such as polyether block amide, polybutylene terephthalate/polybutylene oxide copolymers sold under the Hytrel® and Arnitel® trademarks, Nylon 11, Nylon 12, polyurethane, polyethylene terephthalate, polyvinyl chloride, polyethylene naphthalene dicarboxylate, olefin/ionomer copolymers, polybutylene terephthalate, polyethylene naphthalate, ethylene terephthalate, butylene terephthalate, ethylene naphthalate copolymers, polyetheretherketone, polycarbonates, polyamide/polyether/polyester, polyamides, aromatic polyamides, polyurethanes, aromatic polyisocyanates, polyamide/polyether, and polyester/polyether block copolymers, among others.

In some embodiments, the filter membrane 12 can be formed from at least one of polyether block amide, olefin/ionomer copolymers, nylon, polyurethane, polyethylene terephthalate, polyvinyl chloride, polyethylene naphthalene dicarboxylate and mixtures or copolymers thereof.

The filter membrane 12 can be porous, having pores 14 that are configured to permit blood flow while retaining embolic material of a desired size. The filter membrane 12 can have a mouth 16 and a closed end 18 and is capable of moving between an open state and a closed state. The mouth 16 can be sized to occlude the lumen of the body vessel in which the filter may be installed, thereby directing all fluid and any emboli into the filter with emboli retained therein.

A support hoop 20 can be attached to the filter membrane 12 at or proximate to the mouth 16. The support hoop 20 can be attached to the filter membrane 12 through melt bonding or other suitable means. In some embodiments, as discussed in greater detail hereinafter, the support loop 20 can be integrally molded within the filter membrane 12. The support hoop 20 has an expanded state and a compressed state. The expanded state of the support hoop 20 is configured to urge the mouth 16 to its full size, while the compressed state permits insertion into a small lumen.

The support hoop 20 can be made from a flexible metal such as spring steel, from a super-elastic elastic material such as a suitable nickel-titanium alloy, or from other suitable material. The support hoop 20 can be a closed hoop made from a wire of uniform diameter, it can be a closed hoop made from a wire having a portion with a smaller diameter, it can be an open hoop having a gap, or it can have another suitable configuration.

A strut 22 can be fixedly or slideably attached to and extend from the support hoop 20. An elongate member 24 can be attached to and extend from the strut 22. The elongate member 24 can be attached to the strut 22 at an angle or the strut 22 can have a small bend, either at a point or over a region. The strut 22 can be attached to the support hoop 20 at a slight angle such that when the elongate member 24, the strut 22, and the support hoop 20 are in an unconstrained position, the elongate member 24 can generally extend perpendicular to the support hoop 20.

In the unconstrained position, the elongate member 24 can also lie along an axis which passes through the center of the region created by the support hoop 20. This may help position the support hoop 20 in contact with the wall of a vascular lumen or it may help in enhancing predictability or reliability during deployment. In some embodiments, the elongate member 24 can terminate at the strut 22. In other embodiments, the elongate member 24 can extend through the filter membrane 12, as shown. Whether or not the elongate member 24 extends through the filter membrane 12, it may be fixedly or slideably/rotatably attached to the filter membrane 12.

The filter membrane 12 can include a waist 26 at a closed end 28. In some embodiments, the waist 26 can be integrally formed with the filter membrane 12. In other embodiments, the filter membrane 12 can be further processed to form the waist 26. In some embodiments, integrally forming the waist 26 with the filter membrane 12 can reduce the outer diameter of the filter device when in a compressed state, increase the reliability and uniformity of the bond between the filter membrane and the elongate member, and reduce the number of steps or components needed to form the filter device.

The waist 26 is a region largely incapable of moving between two states and having a lumen of substantially constant diameter therethrough. The elongate member 24 can extend through and be bonded to the waist 26. This bonding can be heat bonding such as laser bonding, or may be an adhesive or other suitable means.

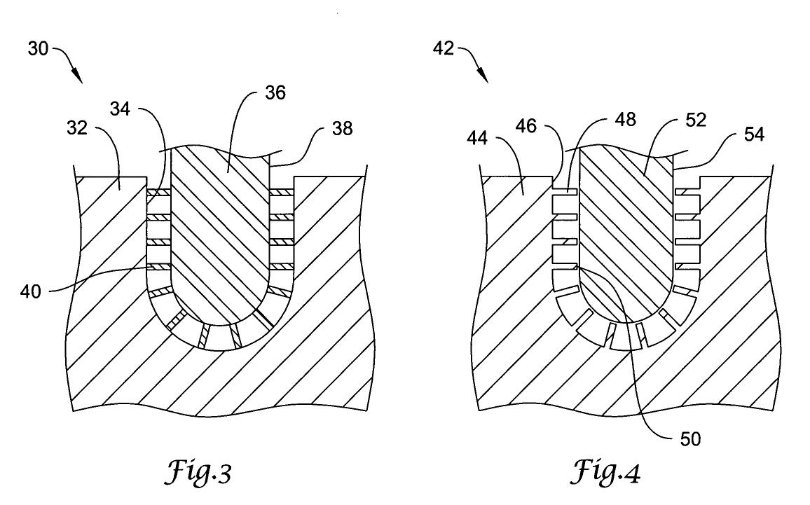

FIG. 3 illustrates a mold assembly 30 that can be used to form the filter membrane 12 described above. The mold assembly 30 includes a mold 32 having a mold surface 34 and a die 36 having a die surface 38. A plurality of protrusions 40 extend between the mold surface 34 and the die surface 38. In some embodiments, the protrusions 40 can be integrally formed with and extend from the mold surface 34. In other embodiments, the protrusions 40 can be integrally formed with and extend from the die surface 38. In some embodiments, it is contemplated that some of the protrusions 40 can extend from the mold surface 34 while others of the protrusions 40 can extend from the die surface.

In other embodiments, the protrusions 40 can be separately formed and then mechanically, thermally or adhesively secured to either the mold surface 34 or the die surface 38. In some embodiments, if the protrusions 40 are formed independently of either the mold 32 or the die 36 to which they will be secured, the protrusions 40 can be attached to either the mold surface 34 or the die surface 38 using an adhesive such as. In other embodiments, the protrusions 40 can be thermally or sonically welded to either of the mold surface 34 or the die surface 38. In some embodiments, the protrusions 40 can be threadedly secured to either of the mold surface 34 or the die surface 38.

The protrusions 40 can be formed having a variety of geometries. In some embodiments, at least some of the protrusions 40 can be cylindrical in shape. In some embodiments, all of the protrusions 40 can be cylindrical. Other suitable geometries include protrusions 40 having an oval, square, rectangular or polygonal cross-section profile. In some embodiments, the protrusions 40 will be cylindrical with a length that ranges from about 0.001 inches to about 0.100 inches and a diameter that ranges from about 0.0005 inches to about 0.0010 inches. The length of the protrusions 40 can, in some embodiments, determine the final thickness of the filter membrane 12.

In some embodiments, at least some of the protrusions 40 can extend from either the mold surface 34 or the die surface 38 in a direction that is substantially perpendicular to either of the mold surface 34 or the die surface 38. In some embodiments, all of the protrusions 40 can extend perpendicularly.

As noted, the mold assembly 30 includes a plurality of protrusions 40. The number of protrusions 40 provided in the mold assembly 30 can vary, depending on the intended use and overall size of the filter membrane 12. For example, if the filter membrane 12 is intended to be used in a portion of a patient's vasculature that has proportionately greater blood flow, it can be advantageous to provide a greater number of pores 14 (FIG. 1) and, thus, a greater number of protrusions 40 would be used in the mold assembly 30. Conversely, if the filter membrane 12 is intended for use in a situation with proportionately less blood flow, or within a relatively smaller vasculature, fewer pores 14 may be needed, and therefore, a reduced number of protrusions 40 can be used.

The mold 32, the die 36 and the protrusions 40 can each be formed of any suitable material that is sufficiently stable and solid at the temperatures necessary to melt the material used to form the filter membrane 12. In some embodiments, the mold 32, the die 36 and the protrusions 40 can be formed of any metallic or high temperature polymer. Specific examples of suitable materials include polymers such as PEEK (polyether ether ketone) and metals such as steel and titanium. Especially useful materials include polyurethanes.

As noted, the protrusions 40 can extend from either of the mold surface 34 or the die surface 38. FIG. 4 illustrates the former while FIG. 5 illustrates the latter. In particular, FIG. 4 shows a mold assembly 42 having a mold 44 and a die 52. The mold 44 has a mold surface 46 and a plurality of integrally formed protrusions 48 extending from the mold surface 46. Each protrusion 48 has a free end 50 closest to the die 52. The die 52 has a die surface 54.

In some embodiments, the free end 50 can at least partially contact the die surface 54 when the die 52 is fully extended into the mold 44. In some embodiments, there will be a small clearance between the die surface 54 and the free end 50 of each protrusion 48. The small clearance can be a distance sufficient to permit easy insertion of the die 52 into the mold 44, while not permitting molten material (discussed hereinafter) to set between the free end 50 and the die surface 54.

In some embodiments, the mold 44, the protrusions 48 and the die 52 can be made of materials having different compressive strengths. For example, if the protrusions 48 extend from the mold surface 46 as shown in FIG. 4, it can be useful for the protrusions 48 to be made of a material that is somewhat softer or lower in compressive strength than the die 52. As a result, the free ends 50 of the protrusions 48 can fully contact the die surface 54, and as a result, the protrusions 48 can slightly deform to ensure more complete contact between the free ends 50 and the die surface 54, thereby reducing or eliminating any molten material that could otherwise solidify therebetween.

The mold 44, the protrusions 48 and the die 52 can be formed of any suitable material and having any suitable dimensions as discussed previously with respect to the elements of FIG. 1. For example, the mold 44 and the die 52 can be formed of steel, while the protrusions 48 can be formed of titanium. In some embodiments, the mold 44 and the protrusions 48 can be formed of titanium, while the die 52 is formed of steel. In other embodiments, the die 52 and the protrusions 48 can be formed of titanium, while the mold 44 is formed of steel.

FIG. 5 illustrates a mold assembly 56 having a mold 58 and a die 62. The mold 58 includes a mold surface 60. The die 62 includes a die surface 64 and a plurality of integrally formed protrusions 66 extending from the die surface 64. Each of the protrusions 66 include a free end 68 closest to the mold surface 60. As discussed with respect to FIG. 4, the free end 68 of each protrusion 66 can at least partially contact the mold surface 60. In some embodiments, there can be a small clearance between the free ends 68 and the mold surface 60. In some embodiments, the clearance distance can be set to nearly zero.

As discussed above with respect to FIG. 1, in some embodiments the filter membrane 12 can include an integrally formed waist 26, while in other embodiments the waist 26 can subsequently be formed after formation of the filter membrane 12. The mold assemblies 30, 42 and 56 discussed previously are directed to embodiments in which the waist 26, if present, is added during processing subsequent to forming the filter membrane 12.

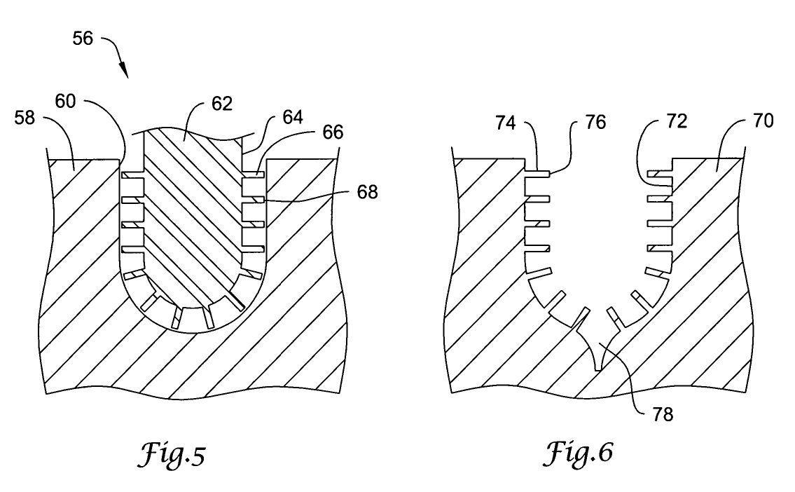

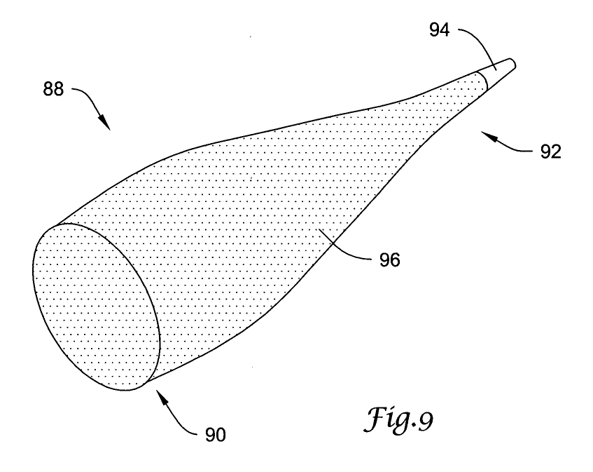

To illustrate an embodiment in which the waist 26 is integrally formed, attention can be turned to FIGS. 6-9. FIGS. 6 through 8 illustrate an embodiment of a mold assembly, while FIG. 9 illustrates a filter membrane produced using this mold assembly.

In particular, FIG. 6 shows a mold 70 having a mold surface 72 and a plurality of integral protrusions 74. Each of the protrusions 74 include a free end 76. The mold 70 includes a tapered portion 78 that is configured to provide the aforementioned waist 26 (FIG. 1).

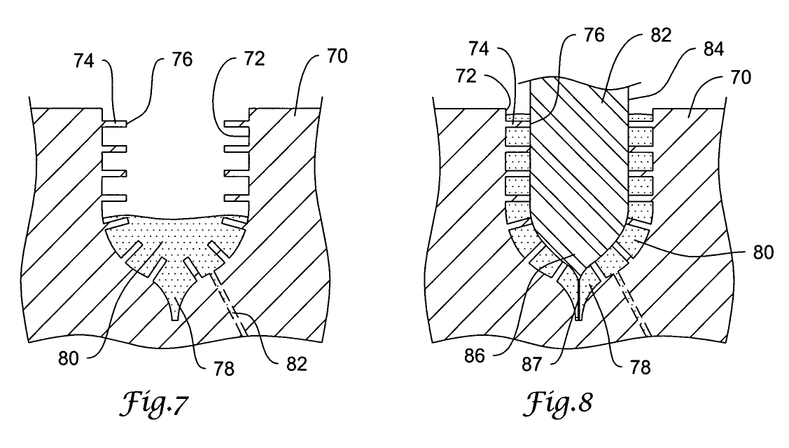

In FIG. 7, a quantity of a molten material 80 has been placed within the mold 70. In some embodiments, the molten material 80 can simply be poured into the mold 70. In other embodiments, the mold 70 may otherwise be sealed. In such circumstances, the molten material 80 can be injected into the mold 70 through, for example, an injection port 82 (seen in phantom). The molten material 80 can be at a temperature that is in the range of about 80° C. to about 200° C.

Once the molten material 80 has been placed in the mold 70, the die 82 can be inserted into the mold 70 as illustrated for example in FIG. 8. The die 82 includes a die surface 84 that at least partially contacts the free ends 76 of the protrusions 74. The die 82 includes a tapered extension 86 and a pin 87 that cooperate with the previously discussed tapered portion 78 of the mold 70 to form a waist 26 (FIG. 1). The pin 87 assists in forming an axially aligned aperture through the waist 26 that can be sized to accommodate a guidewire. As the die 82 is inserted into the mold 70, the molten material 80 is forced upwards to fill the spaces between and around the protrusion 74, the mold surface 72 and the die surface 84.

In some embodiments, it can be useful to apply at least a portion of the molten material 80 to the die surface 84 prior to inserting the die 82 into the mold 70. A portion of the molten material 80 can be sprayed or coated onto the die surface 84. In some embodiments, the die 82 can be dipped into a supply of the molten material 80 prior to inserting the die 82 into the mold 70. Depending on the viscosity and other properties of the molten material 80, it may be useful to mechanically assist distribution of the molten material 80 within the mold 70. In some embodiments, it can be useful to agitate or spin at least one of the mold 70 and the die 82.

Once the molten material 80 solidifies, the mold 70 and the die 82 can be separated to free the resulting filter membrane 88 illustrated in FIG. 9. Depending on the clearance between the mold surface 72 and the free ends 76 of the protrusions 74, a small amount of solidified material may be present between the mold surface 72 and the free ends 76, effectively blocking the apertures otherwise formed by the protrusions 74. In some embodiments, it can be useful to vibrate either the mold 70 or the die 82 with respect to the other of the mold 70 and the die 82 in order to remove this material and open the apertures.

The filter membrane 88 includes a proximal region 90 and a distal region 92 including an integrally formed waist 94. The filter membrane 88 includes a plurality of integrally molded apertures 96 configured to selectively pass blood and other similar fluids while impeding undesirable material such as embolic material.

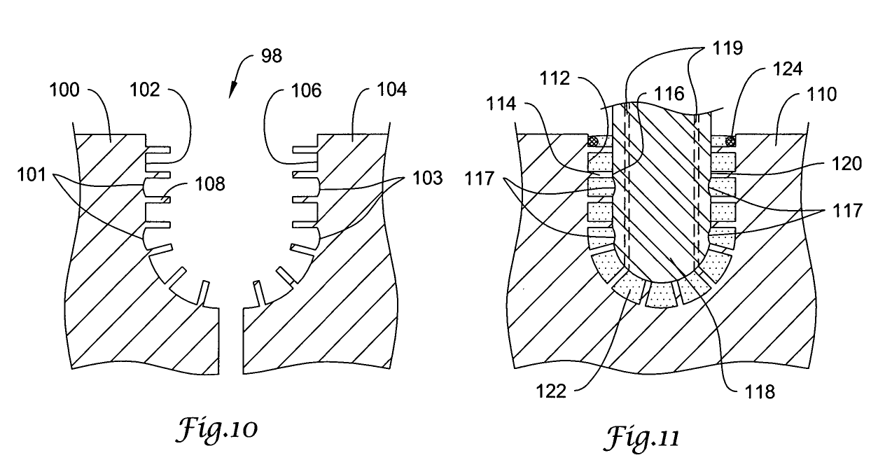

FIG. 10 illustrates a particular embodiment of mold 98 that includes a first mold section 100 having a first mold surface 102 and a second mold section 104 having a second mold surface 106. A plurality of protrusions 108 extend from both the first mold surface 102 and the second mold surface 104 as previously discussed. In this embodiment, once the molten material 80 has solidified, the mold 98 can be separated into two distinct mold sections 100 and 104 in order to facilitate removal of the filter membrane 88.

In some embodiments, it can be useful to provide one or more reinforcing ribs (not illustrated in FIG. 9) in the filter membrane 88. In FIG. 10, the first mold surface 102 and the second mold surface 104 each include one or more annular grooves 101 and 103, respectively. The annular grooves 101 and 103 will permit the formation of radially oriented reinforcing ribs that are positioned on or near an external surface of the filter membrane 88.

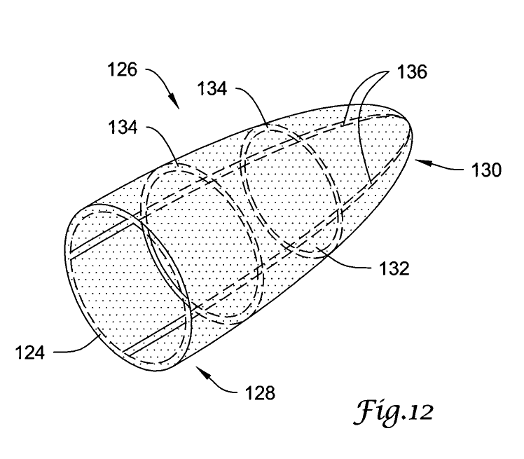

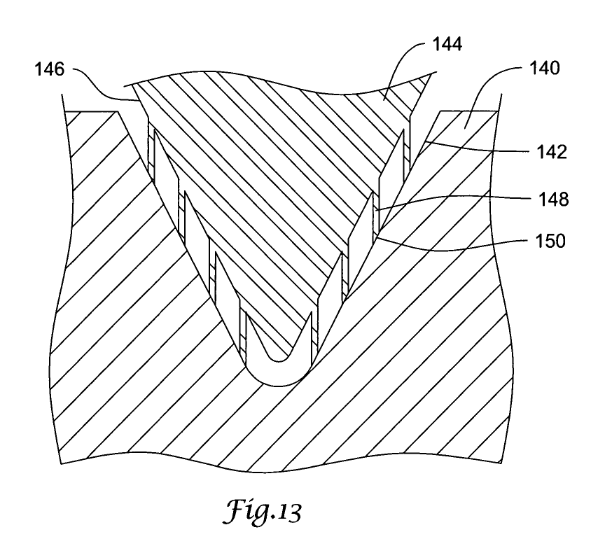

FIG. 11, however, provides provision for forming reinforcing ribs that are positioned on or near an interior surface of the filter membrane 126 (see FIG. 12). In FIG. 11, the die 118 includes at least one radially oriented annular groove 117 and at least one axially oriented groove 119.

FIG. 12 shows that the filter membrane 126 includes at least one radially oriented reinforcing rib 134 and at least one axially oriented reinforcing rib 136. Using the mold and die assembly described in FIG. 11 will result in reinforcing ribs 134 and 136 that are positioned at or near an interior surface of the filter membrane 126.

Moreover, FIGS. 11 and 12 illustrate a particular embodiment in which a support loop is integrally molded into a filter membrane. FIG. 11 shows a mold 110 having a mold surface 112 and a plurality of protrusions 114 extending from the mold surface 112. Each of the protrusions 114 has a free end 116. A die 118 having a die surface 120 is seen inserted into the mold 110.

Previous to die insertion, a quantity of molten material 122 is placed within the mold 110, and a support loop 124 is placed into the mold 110. Once the die 118 has been fully inserted into the mold 110 (as illustrated) such that the die surface 120 is at least partially in contact with the free ends 116 of the protrusions 114, the molten material 122 flows upward to fill in the spaces between and around the mold surface 112, the die surface 120 and the protrusions 114. Once the molten material 122 solidifies, the resulting filter membrane 126 (FIG. 12) can be removed.

As illustrated in FIG. 12, the filter membrane 126 has a proximal region 128 and a distal region 130. The proximal region 128 includes the support loop 124 that is integrally molded into the filter membrane 126. The filter membrane 126 includes a plurality of apertures 132 that are sized and configured to permit blood flow therethrough.

In some embodiments, it may be useful for the apertures formed in the filter membrane to be more closely aligned with blood flow through the particular vasculature in which the filter membrane will be deployed. FIG. 13 shows a mold 140 having a mold surface 142 and a die 144 having a die surface 146. The die 144 includes a plurality of protrusions 148 that extend from the die surface at an angle that positions the protrusions 148 at least approximately parallel to a long axis of the die 144. In some embodiments, the protrusions 148 could, instead, extend from the mold surface 142. As a result, the apertures that will be formed in the filter membrane resulting from use of this mold 140 and die 144 will be more closely aligned with blood flow.

In some embodiments, it can be useful for the apertures to have an ovoid cross-sectional profile. As a result of having an ovoid shape, the apertures can provide a more direct flow path through the apertures, even though the apertures may be formed perpendicular or substantially perpendicular to the surface of the mold.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

1. A method of forming a filter membrane employing a mold assembly, the mold assembly comprising a mold having a mold surface, a die having a die surface and a plurality of protrusions extending from at least one of the mold surface and the die surface, the method comprising steps of:
placing a molten material within a portion of the mold;
inserting the die into the mold such that the protrusions span a distance between the mold surface and the die surface; and
allowing the molten material to solidify, thereby forming the filter membrane, the filter membrane including a plurality of integrally formed apertures.

2. The method of claim 1, wherein when the die is inserted into the mold, the protrusions extending from one of the mold surface or the die surface at least partially contact the other of the mold surface or the die surface.

3. The method of claim 2, wherein the distance between the mold surface and the die surface determines a desired thickness of the filter membrane.

4. The method of claim 1, wherein placing a molten material comprises placing a molten material selected from the group consisting of polyether block amide, olefin/ionomer copolymers, nylon, polyurethane, polyethylene terephthalate, polyvinyl chloride, polyethylene naphthalene dicarboxylate and mixtures or copolymers thereof.

5. The method of claim 1, further comprising a step of agitating the mold to improve molten material distribution.

6. The method of claim 5, wherein agitating the mold is subsequent to extending the die into the mold.

7. The method of claim 1, further comprising a step of spinning the mold to improve molten material distribution.

8. The method of claim 7, wherein spinning the mold is subsequent to extending the die into the mold.

9. The method of claim 1, further comprising a step, subsequent to allowing the molten material to solidify, of vibrating the die or the mold in order to remove material located between the die surface or the mold surface and the protrusions.

10. The method of claim 1, further comprising the step of opening the mold to remove the filter membrane.

11. The method of claim 10, wherein opening the mold comprises withdrawing the die.

12. The method of claim 10, wherein the mold comprises two mold portions, and opening the mold comprises separating the two mold portions.

13. The method of claim 1, wherein providing the molten material comprises pouring the molten material into the mold.

14. The method of claim 1, wherein providing the molten material comprises injecting the molten material into the mold.

15. The method of claim 1, wherein providing the molten material further comprises a step of coating, spraying or dipping the die prior to extending the die into the mold.

16. The method of claim 1, wherein the protrusions comprise cylindrical or ovoid protrusions.

17. An assembly for forming a filter membrane, comprising:
a mold comprising a mold surface defining an at least partially conical cavity;
a plurality of protrusions extending outwardly from the mold surface, each of the protrusions having a protrusion length and a free end, in combination defining a cavity surface; and
a die comprising a die surface complementary to the cavity surface;
wherein when the die is inserted into the mold, the protrusions extending outwardly from the mold surface contact the die surface.

18. The assembly of claim 17, wherein at least most of the protrusions extend from the mold surface.

19. The assembly of claim 18, wherein at least most of the protrusions are at least substantially perpendicular to the mold surface.

20. The assembly of claim 18, wherein at least most of the protrusions extend from the mold surface at an angle sufficient to position the protrusions parallel to an axis of the mold.

21. The assembly of claim 17, wherein at least most of the protrusions extend from the die surface.

22. The assembly of claim 21, wherein at least most of the protrusions are at least substantially perpendicular to the die surface.

23. The assembly of claim 21, wherein at least most of the protrusions extend from the die surface at an angle sufficient to position the protrusions parallel to an axis of the mold.

24. The assembly of claim 17, wherein the protrusion length is set equal to a desired membrane thickness.

25. The assembly of claim 17, wherein at least some of the protrusions are cylindrical.

26. The assembly of claim 21, wherein at least some of the protrusions are ovoid.

27. The assembly of claim 25, wherein each of the protrusions have a length that is in the range of about 0.001 inches to about 0.010 inches and a diameter that is in the range of about 0.001 inches to about 0.010 inches.

28. The assembly of claim 17, wherein the mold surface comprises at least one annular groove configured to provide a radially oriented reinforcing rib in a filter membrane produced using the assembly.

29. The assembly of claim 17, wherein the mold surface comprises at least one axially oriented groove configured to provide an axially oriented reinforcing rib in a filter membrane produced using the assembly.

30. The assembly of claim 17, wherein the die surface comprises at least one annular groove configured to provide a radially oriented reinforcing rib in a filter membrane produced using the assembly.

31. The assembly of claim 17, wherein the die surface comprises at least one axially oriented groove configured to provide an axially oriented reinforcing rib in a filter membrane produced using the assembly.

32. A filter membrane formed by a process comprising steps of:
providing a mold, the mold comprising a mold surface and a plurality of protrusions extending outwardly from the mold surface to define a cavity surface;
providing a complementary die, the die comprising a die surface;
providing a molten material within a portion of the mold; and
extending the die into the mold such that the protrusions contact the die surface; and
permitting the molten material to solidify, thereby forming the filter membrane, the filter membrane comprising a plurality of apertures.

33. The filter membrane of claim 32, wherein the process further comprises a subsequent step of withdrawing the die from the mold to free the filter membrane.

34. The filter membrane of claim 32, wherein the plurality of apertures are integrally molded into the filter membrane and are sized to permit blood to pass through the apertures but not permit embolic material to pass through the apertures.

35. The filter membrane of claim 32, wherein the molten material is selected from the group consisting of polyether block amide, olefin/ionomer copolymers, nylon, polyurethane, polyethylene terephthalate, polyvinyl chloride, polyethylene naphthalene dicarboxylate and mixtures or copolymers thereof.

36. The filter membrane of claim 32, wherein as a result of the process used to form the filter membrane, the filter membrane is conical in shape and has a uniform membrane thickness.

37. The filter membrane of claim 32, wherein as a result of the process used to form the filter membrane, the integrally formed apertures are formed parallel to an axis of the filter membrane.

38. A filter assembly comprising:
a support loop;
a filter membrane having a proximal region and a distal region, the support loop integrally molded into the proximal region of the filter membrane, the filter membrane having a substantially constant thickness;
a distal waist positioned proximate the distal region of the filter membrane; and
a plurality of integrally formed apertures within the filter membrane.

39. The filter assembly of claim 38, further comprising one or more radially oriented reinforcing ribs integrally molded into the filter membrane.

40. The filter assembly of claim 38, further comprising one or more axially oriented reinforcing ribs integrally molded into the filter membrane.

41. The filter assembly of claim 38, wherein the integrally formed apertures are ovoid.

42. The filter assembly of claim 38, wherein the integrally formed apertures have a cross section profile and a length, and the apertures are positioned such that the length is parallel to an axis of the filter assembly.
